(19) United States
(12) Patent Application Publication
(10) Pub. No.: US 2021/0204862 A1
(43) Pub. Date: Jul. 8, 2021

(54) ELECTROGUSTOMETER

(71) Applicant: MY ROBOTICS, Paris (FR)

(72) Inventor: Pierre-Antoine BASTIAN, Paris (FR)

(73) Assignee: MY ROBOTICS, Paris (FR)

(21) Appl. No.: 16/650,399

(22) PCT Filed: Sep. 21, 2018
(86) PCT No.: PCT/FR2018/052303

§ 371 Date: Mar. 25, 2020

(30) Foreign Application Priority Data
Sep. 25, 2017 (FR) 1758832

(51) Int. Cl. A61B 5/00

(52) U.S. Cl. CPC A61B 5/4017; A61B 5/7225; A61B 5/7264; A61B 2560/02; A61B 5/4082; A61B 2560/0475; A61B 5/4088

(57) ABSTRACT

An electrogustometer comprises an interface (4) capable of receiving a stimulation instruction, and a current generator (8) arranged to produce a stimulation current. The interface (4) is arranged to obtain a digital command from the stimulation instruction, and the electrogustometer further comprises a digital-to-analogue converter (6) suitable for converting the digital command into a DC voltage, the different digital commands inducing discrete respective voltage values. The current generator (8) is arranged to convert the DC voltage into a stimulation current with a strength of between 0 and 250 μA, depending on the discrete voltage value, and varies by fixed steps of between 0.05 μA and 1 μA.

### FIELD OF THE INVENTION

The invention relates to the field of gustometry, and in particular of electrogustometry.

### BACKGROUND

Taste is a complex sense the mechanisms whereof are relatively little-known. Nevertheless, taste disturbance, when quantified, may be very useful in medical follow-up, for example in the follow-up of oncology patients, prevention and support for cessation in the treatment of tobacco use and early diagnosis of certain diseases, for example Alzheimer's and Parkinson's disease.

Gustometry aims to analyze taste disturbances, by analyzing a subject's perception of taste by eliminating a smell disorder. Three categories of procedures are available to physicians: chemical gustometry, electrogustometry and biopsy of the papillae to analyze taste bud tissue.

Chemical gustometry consists of depositing one or more chemical substances on a patient's tongue and measuring their reaction(s). Electrogustometry consists of measuring the detection of taste by a patient by means of the application of a very low current on their tongue, the aim being to measure the lowest possible current that will induce the onset of the taste that a subject is capable of perceiving.

Chemical gustometry is impractical for numerous reasons: the patient's reaction time, the patient's relaxation time, difficulty in dosing, applying repeatable procedures, etc. To this is added the need to have a chemical laboratory, the long preparation time, restrictive chemical substance storage conditions, etc.

Electrogustometry, through use of electricity, would appear at first glance to have more potential. Yet, to date, the Applicant is only aware of a single electrogustometry apparatus which appears to have been the subject of commercial production, the TR-06 Rion manufactured by the company Sensonics, INC.

It consists of a relatively rudimentary apparatus, wherein the current applied is delivered between 4 μA and 400 μA with a mean discretisation step of 20 μA.

The application of a stimulus is solely manual, controlled via a pedal assembly to activate the dispatch of current, and the operator notes on paper the stimulation parameters applied to the patient before making the electrogustometric threshold calculations themselves. The patient is given a certain recovery time for the sour taste from a stimulation to disappear, but this time is dependent on the user. Similarly, the stimulation time is only programmable to at most 2 seconds of stimulation time, while a patient's reaction time is very widely variable. The TR-06 Rion is cumbersome, and has a total weight of 2.3. kg, making it a heavy, bulky, and difficult-to-transport apparatus. Due to the complexity of the operation thereof and the lack of automation of the TR-06 Rion, the use thereof is currently purely clinical and exploratory, without it being possible to envisage the daily use thereof by a physician with their patients.

The scale thereof renders it very imprecise, the size thereof, the complex handling thereof and the technological limitations thereof render it very impractical, so much so that this device does not appear to be actually used in practice, including for clinical studies. In this regard, the abstract of the article “A comparison of two electric taste stimulation devices” by McClure S T et al, Physiol Behav. Nov. 23, 2007; 92(4):658-64, states, when comparing the TR-06 Rion to an improvised electrogustometer with a 1.6V battery concludes as follows: “The battery device may provide an inexpensive portable alternative to an electrogustometer for use in clinical testing of taste”.

For these reasons, electrogustometry is extremely underdeveloped to date, and even replaced by chemical gustometry despite the disadvantages described above. There is therefore a genuine need to offer a medical electrogustometer, which can be used by physicians daily with their patients, in the context of protocols enabling follow-up of the patients' perception of taste, as well as in the context of dosage adaptation, prevention, support for cessation or disease diagnosis.

### SUMMARY

For this purpose, the invention relates to an electrogustometer comprising an interface capable of receiving a stimulation instruction, and a current generator arranged to produce a stimulation current. The interface is arranged to obtain a digital command from the stimulation instruction, and the electrogustometer further comprises a digital-to-analogue converter suitable for converting the digital command into a DC voltage, the different digital commands inducing discrete respective voltage values, and the current generator is arranged to convert the DC voltage into a stimulation current with an intensity of between 0 and 250 μA, depending on the discrete voltage value, and varies by fixed steps of between 0.05 μA and 1 μA.

This electrogustometer is advantageous as it makes it possible to carry out reliable gustometry protocols. Furthermore, due to the precision thereof, it may be used in a medical environment on a day-to-day basis. Indeed, the features thereof enable this electrogustometer to be very precise (within 1% in relation to that which the user wishes to send as current), modulable according to the user's needs and comply with the regulatory constraints that enable it to be certified as a type IIa Medical Device. Thus, there is no risk of sending too much current to the patient's tongue, or of making an error in the electrogustometric threshold measurement calculations, while making it possible to carry out a taste sensitivity test in scarcely a few minutes.

In various alternative embodiments, the electrogustometer according to the invention may have one or more of the following features:
the current generator comprises a bipolar transistor assembly,
the electrogustometer further comprises an electrode for applying a stimulation current generated by the current generator,
the interface comprises an offset electronic device connected to a microcontroller,
the offset electronic device comprises a process for automatically determining an electrogustometric threshold based on one or more feedbacks in response to the application of a series of stimulation currents generated by the current generator,
the electronic device comprises a process for automatically determining an electrogustometric threshold based on Dixon's algorithm,
the electronic device is arranged to use a double-blind process,
the offset electronic device is arranged to use a process wherein the application of a series of stimulation currents generated by the current generator comprises the emission of stimulation currents for a fixed time, from an initial intensity value, each stimulation current being separated from the preceding emission by a fixed intensity value,
the electronic device comprises a process for automatically determining an electrogustometric threshold based on a dichotomic progression, and
the digital-to-analogue converter receives a digital command in the form of a number, and in that each discrete voltage value corresponds to a digital command.

### BRIEF DESCRIPTION OF THE DRAWINGS

Further features and advantages of the invention will emerge more clearly on reading the following description, based on examples given by way of illustration and not limitation, based on the drawings wherein:

FIG. 1 represents a schematic view of a medical electrogustometer according to the invention,

FIG. 2 represents a schematic electrical diagram of an element in FIG. 1,

FIG. 3 represents an example of use of a function by an element in FIG. 1, and

FIG. 4 represents an example of use of a function in an alternative embodiment by an element in FIG. 1.

### DETAILED DESCRIPTION OF THE PREFERRED EMBODIMENTS

The drawings and the description hereinafter contain, essentially, elements of certain characteristics. Therefore, they will be able to serve not only to create a better understanding of the present invention, but also contribute to the definition thereof, where necessary.

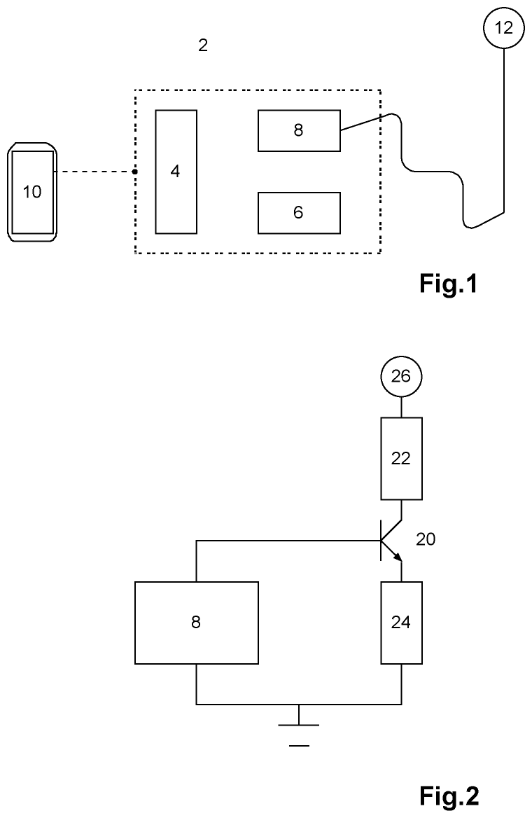

FIG. 1 represents a schematic diagram of an electrogustometer 2 according to the invention.

The electrogustometer 2 comprises a microcontroller 4, a digital-to-analogue converter 6 and a current generator 8.

The microcontroller 4 receives stimulation intensity instructions from a telephone 10, and the current generator 8 emits a current to an electrode 12 in response. Thus, with the telephone 10, the electrogustometer 2 and the electrode 12, it is possible to conduct an electrogustometry procedure by arranging the element 12 on the patient's tongue and collecting the responses from the latter to stimulations.

In the example described herein, the microcontroller 4 is of Microchip brand ATMegal168 type. The microcontroller 4 is arranged to receive stimulation instructions from the telephone 10 and to convert them into digital commands. The microcontroller 4 therefore acts as an interface for controlling the intensity of the current emitted by the electrogustometer 2. The telephone 10 is in the example described herein of the smartphone type and communicated via a wireless protocol with the microcontroller 4 such as Bluetooth. Other wireless protocols could be used, and the telephone 10 could be connected by a cable (for example USB) to an input of the microcontroller 4. The telephone 10 could be replaced by a tablet or by another offset electronic device suitable for executing an algorithm as described hereinafter and suitable for communicating with the interface of the electrogustometer 2. Alternatively still, the telephone 10 could be integrated in the electrogustometer 2 so as to offer a directly accessible interface. The conversion of the stimulation instruction into a digital command may be based on a correspondence rule, on a look-up table type search table, or on a direct command with a feedback loop.

FIG. 2 represents an example of an electrical diagram used by the current generator 8 to convert the voltage emitted at the output of the digital-to-analogue converter 6 into current for the electrode 12.

As can be seen in this figure, the output of the digital-to-analogue converter 8 is connected to the base of a bipolar transistor 20. The emitter of the transistor 20 is connected in series with a resistor 22 which represents the resistance of the patient's tongue when the latter is connected to the electrode 12, whereas the collector of the transistor 20 is connected in series to a resistor 24 which is grounded. The resistor 22 is also in series with the other part of the electrode 12 which is in series with a DC voltage source 26. To close the circuit when a stimulation current is emitted, the person receiving the electrode 12 wears a wristband (not shown in FIG. 2) which is connected to the ground downstream from the resistor 24.

According to this diagram, the intensity passing through the resistor 22 is the intensity ic at the level of the collector of the transistor 20. Once the voltage between the collector and the emitter of the transistor 20 remains positive, the intensity ic is substantially equal to the intensity ie at the level of the emitter.

Thus, the intensity ic is equal to the difference between the output voltage of the digital-to-analogue converter 6 and the voltage between the collector and the base of the transistor 20, divided by the resistor 24.

In the example described herein, the digital-to-analogue converter 6 is of the MCP4725 from the company Microchip. The use of an analogue-to-digital converter is particularly advantageous as it makes it possible to control with precision the voltage output thereby. Thus, in combination with the current generator 8, it is possible to control the intensity of the current emitted to the electrode 12 with a precision of 0.1 μA.

Alternatively, the current generator 8 could also be based on a field-effect transistor circuit, or on operational amplifier assembly by means of suitable cooling.

The Applicant discovered that the electrogustometer 2 described with FIGS. 1 and 2 is particularly advantageous as it makes it possible to use algorithms suitable analyzing the patient's electrogustometric threshold rapidly, automatically and with precision.

Thus, the Applicant discovered that Dixon's algorithm described in the book by Dixon & Massey “Introduction to Statistical Analysis”, Mac Graw Hill in 1960. Historically, Dixon's algorithm was developed to determine with precision the quantity of explosive to explode a chosen quantity of rock. Although there is no direct link with electrogustometry, the Applicant discovered that applying Dixon's algorithm with the electrogustometry according to the invention gives extremely precise results, extremely rapidly and reliably.

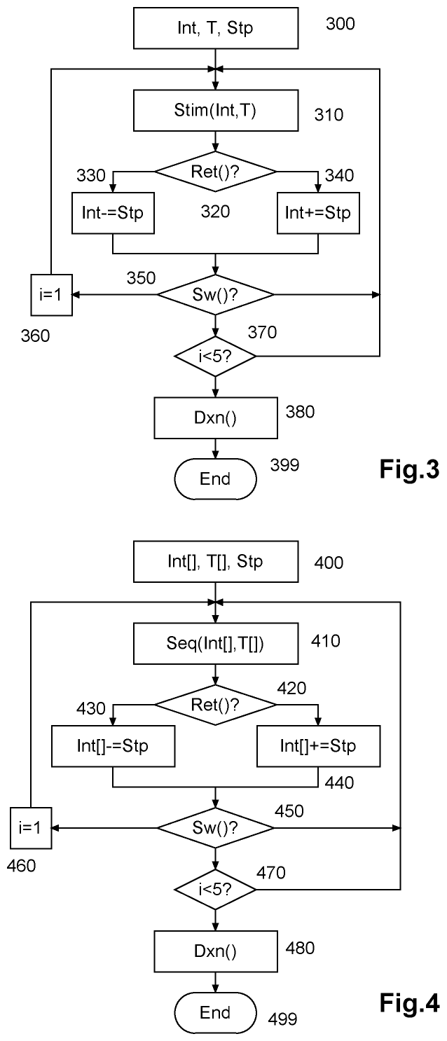

FIG. 3 represents an example of use of a first version of Dixon's algorithm with the electrogustometer 2. In an operation 300, the initial intensity Int is set, along with the step Stp and the stimulation time T. These parameters may for example be introduced via an application executed by the telephone 10 and which will control the electrogustometer 2. The telephone 10 may be held by the user, who will then ask the patient whether they have perceived the stimulation or not. This makes it possible to avoid influencing the patient psychologically and discourage the patient from lying about their perception.

Once the parameters have been set, a stimulation loop is executed. In an operation 310, the telephone 10 executes a function Stim( ) which instructs the electrogustometer 2 to emit a direct current of intensity Int for a time T to the electrode 12.

In response to the stimulation, the patient indicates in an operation 320 whether they have perceived the stimulation or not. If the patient indicates having perceived a stimulation, then the intensity Int is decremented by the step Stp in an operation 330. If the patient indicates not having perceived a stimulation, then the intensity is incremented by the step Stp in an operation 340.

A function Sw( ) is then executed in an operation 350. This function compares the result of the current loop with that of the preceding loop. If the patient's response is the same, then the loop resumes with the operation 310. Otherwise, a shift has been performed, and 4 additional measurements will be carried out to characterise the measurement.

Thus, in an operation 360, an index i is set to the value 1. Then, in all the executions of the loop, and until the end of the function, the function Sw( ) will no longer perform a test and will confine itself to incrementing the index i. This may for example be performed by activating a flag on the detection of the shift by the function Sw( ), the activated flag triggering the bypass of the test in the function Sw( ) and the incrementation of the index i. Further alternative embodiments are obviously possible.

Finally, a test is carried out in an operation 370 in order to determine whether the 4 operations following the detection of the shift have been performed or not. If so, then the user's responses on the last 5 measurements are used jointly with the Dixon matrix to calculate the patient's taste threshold by means of a function Dxn( ) in an operation 380, and the function finishes in an operation 399. Otherwise, the loop resumes with the operation 310 for the last measurements to be carried out.

FIG. 4 represents a function in an alternative embodiment of FIG. 3 wherein Dixon's algorithm is still used, but on the basis this time of a double-blind test. For this, the stimulation and measurement are slightly different in that two stimulations will be proposed each time to the patient, of which only one will be real. The response that will be requested of the patient relates to that of the two stimulations which actually occurred.

Thus, the operations of the function of FIG. 4 are very similar to those of the function of FIG. 3. They differ in that:
an intensity Int is no longer involved, but a vector Int[ ] which receives the intensity of the first and the second stimulation, as well as for the time T which is a vector T[ ]. The values of Int[ ] are only Int and 0, and the time is the same for both stimulations,
the operation 410 is no longer a direct stimulation, but the execution of a sequence by a function Seq( ) which carries out the double-blind function, i.e. only the application in the telephone 10 knows which of the first or the second stimulation has been performed with the intensity Int for a given loop execution, and
the operation 420 is not based on the patient's response alone, but on the comparison between their response and the reality known by the application. Thus, if the patient has correctly identified the stimulation carried out, then the intensity is decremented, whereas in the opposite case it is increased.

Further test algorithms could be used via an application on the telephone 10, such as an algorithm based on dichotomy, or other. The application may also enable a user to control the electrogustometer 2 directly, without passing via an algorithm. In this case, the intensity and the time are specified directly on the telephone 10 and, as in all the protocols, the patient is then requested by the user whether they have perceived the stimulation or not. Alternatively, the patient may use the electrogustometer and the application alone, with a simplified measurement protocol. In this case, a “STOP” type button is displayed on the screen of the mobile application, that the patient holds with one hand, and, with the other hand, they hold the electrode positioned on the tongue. As long as the patient does not press the “STOP” button, the current increases gradually on the electrogustometer and sends a stimulation always having the same time and the same incrementation step. Once the patient presses “STOP”, the result is displayed on the application. If the current reaches the maximum intensity, the test is stopped, and an alert message is displayed on the smartphone and the user may be prompted to start again.

Optionally, for each protocol, if the electrode is disconnected from the patient's tongue, then an error message is displayed, the stimulation is stopped and the user may resume the test with the same value as previously.

The electrogustometer 2 described herein is especially advantageous as it is naturally integrable in a connected environment. Indeed, as it is no longer only manually controllable according to a protocol used by a researcher, but by a modern electronic interface, it may naturally be integrated in a connected environment, and enable easy integration of a patient's measurement results into all of their medical data. This will make it possible to create the first database on taste disturbances, and eventually link up taste disturbances and other health data obtained from connected health records and other medical devices, and link up taste disturbance and medical treatments with analysis of treatment side-effects, etc.

What is claimed is:

1. Electrogustometer comprising:
an interface (4) capable of receiving a stimulation instruction, and
a current generator (8) arranged to produce a stimulation current,
wherein the interface (4) is arranged to obtain a digital command from the stimulation instruction,
the electrogustometer further comprises a digital-to-analogue converter (6) suitable for converting the digital command into a DC voltage, the different digital commands inducing discrete respective voltage values,
and the current generator (8) is arranged to convert the DC voltage into a stimulation current with an intensity of between 0 and 250 μA depending on the discrete voltage value and varies by fixed steps of between 0.05 μA and 1 μA.

2. The electrogustometer according to claim 1, wherein the current generator (8) comprises a bipolar transistor assembly.

3. The electrogustometer according to claim 1, further comprising an electrode (12) for applying a stimulation current generated by the current generator (8).

4. The electrogustometer according to claim 1, wherein the interface (4) comprises an electronic device (10) connected to a microcontroller (4).

5. The electrogustometer according to claim 4, wherein the electronic device (10) comprises a process for automatically determining an electrogustometric threshold based on one or more feedbacks in response to the application of a series of stimulation currents generated by the current generator (8).

6. The electrogustometer according to claim 5, wherein the electronic device (10) comprises a process for automatically determining an electrogustometric threshold based on Dixon's algorithm.

7. The electrogustometer according to claim 6, wherein the electronic device (10) is arranged to use a double-blind process.

8. The electrogustometer according to claim 4, wherein the electronic device (10) is arranged to use a process wherein the application of a series of stimulation currents generated by the current generator (8) comprises the emission of stimulation currents for a fixed time, from an initial intensity value, each stimulation current being separated from the preceding emission by a fixed intensity value.

9. The electrogustometer according to claim 5, wherein the electronic device (10) comprises a process for automatically determining an electrogustometric threshold based on a dichotomic progression.

10. The electrogustometer according to claim 1, wherein the digital-to-analogue converter (6) receives a digital command in the form of a number, and each discrete voltage value corresponds to a unique digital command.
